(19) United States
(12) Patent Application Publication
(10) Pub. No.: US 2011/0301201 A1
(43) Pub. Date: Dec. 8, 2011

(54) DABIGATRAN FOR PERCUTANEOUS INTERVENTIONAL CARDIAC CATHETERISATION

(75) Inventor: Paul Anthony Reilly, Danbury, CT (US)

(73) Assignee: BOEHRINGER INGELHEIM PHARMACEUTICALS INC., Ridgefield, CT (US)

(21) Appl. No.: 13/058,920

(22) PCT Filed: Aug. 17, 2009
(86) PCT No.: PCT/EP2009/060592

§ 371 Date: Mar. 15, 2011

Related U.S. Application Data
(60) Provisional application No. 61/090,018, filed on Aug. 19, 2008.

(51) Int. Cl. A61K 31/4439; A61P 7/02

(52) U.S. Cl. 514/338

(57) ABSTRACT

The invention relates to a new use of dabigatran etexilate of formula (I) optionally in the form of the pharmaceutically acceptable salts thereof, and new medicament formulations which may be used for this purpose.

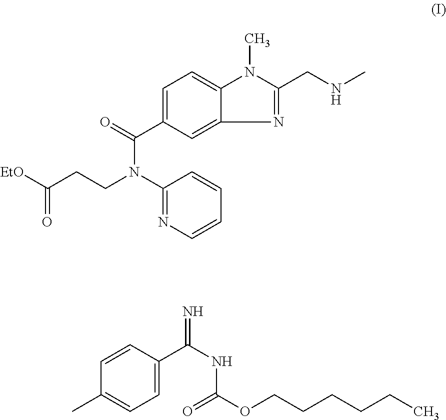

The invention relates to a new use of dabigatran etexilate of formula I

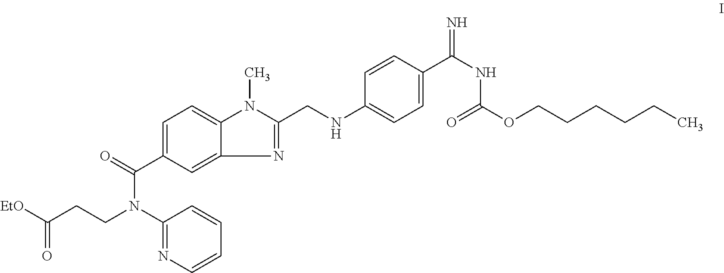

optionally in the form of the pharmaceutically acceptable salts thereof, as well as new medicament formulations which may be used for this purpose.

### BACKGROUND TO THE INVENTION

The compound of formula I is known from the prior art and was first disclosed in WO98/37075. It is a potent thrombin inhibitor which can be used for example for the post-operative prevention of deep vein thromboses and in stroke prevention, particularly for preventing strokes in patients with atrial fibrillation.

The present invention relates to the use of the compound of formula I as a secondary medication in percutaneous interventional cardiac catheterisation.

### DETAILED DESCRIPTION OF THE INVENTION

Percutaneous interventional cardiac catheterisation (PCI=percutaneous coronary intervention) is a test carried out on patients at potential risk of coronary disease. Frequently a symptom such as, for example, a feeling of tightness or pain in the chest or a pathological change in the heart current patterns (electrocardiogram) is the basis for cardiac catheterisation. In this investigation, it is determined whether the coronary blood vessels have flow problems. If any such flow problems are found, percutaneous balloon dilatation or the implanting of a stent in the lestion responsible may be carried out during the cardiac catheterisation.

The present invention relates to the use of the compound of formula I

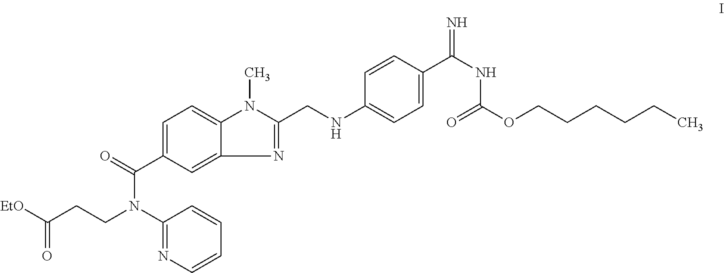

optionally in the form of the tautomers and the pharmaceutically acceptable salts thereof, as a secondary medication in percutaneous, interventional cardiac catheterisation.

Pharmaceutically acceptable salts of dabigatran etexilate include acid addition salts which are selected from among the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydrolactate, hydroxalate, hydrosuccinate, hydrobenzoate and hydro-p-toluenesulphonate, preferably hydrochloride, hydrobromide, hydrosulphate, hydrophosphate, hydromaleate, hydrofumarate and hydromethanesulphonate. The salts of hydrochloric acid, methanesulphonic acid, maleic acid, benzoic acid and acetic acid are particularly preferred. Of exceptional importance according to the invention are the salts of methanesulphonic acid, which are optionally also referred to as mesylates within the scope of the present invention.

The acid addition salts of dabigatran etexilate, particularly the methanesulphonic acid salt, are disclosed for example in WO 03/074056. The specific polymorphs I and II of the methanesulphonic acid salt or the hemihydrate thereof are also known from the prior art (WO 2005/028468). The present invention includes the use of the solvates and hydrates of the salts of the compound of formula I.

The active ingredient of the compound of formula I is called dabigatran and is represented by the following formula II

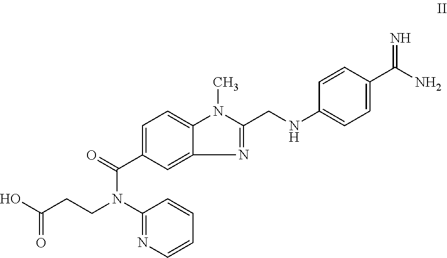

The use according to the invention also includes the use of the compound of formula II as secondary medication in percutaneous, interventional cardiac catheterisation.

Preferably, between 50 and 400 mg, particularly preferably 75 to 350 mg of the compound of formula I are administered per day in order to implement the medication according to the invention. Particularly preferably, 110-300 mg, more preferably 150-220 mg of compound I are administered per day.

The compound of formula I is preferably administered with or before the percutaneous interventional cardiac catheterisation. Particularly preferably the compound of formula I is administered before the cardiac catheterisation. It is advisable for example to start the administration of the compound of formula I in the above-mentioned doses 12-48 h, preferably 16-36 h, particularly preferably 18 to 24 h before the percutaneous interventional cardiac catheterisation.

The compound of formula I is preferably administered using multiparticulate medicament formulations as described for example in WO 03/074056. FIG. 1 of WO 03/74056 shows the schematic structure of preferred pharmaceutical compositions by means of a section through a suitable pellet. The approximately ball-shaped/spherical core region of this pellet contains or consists of a pharmaceutically acceptable organic acid, preferably tartaric acid. Then comes a layer that separates the acid core from the layer containing the active substance, the so-called isolating layer. The isolating layer is in turn surrounded by the active substance layer, which is also in the shape of a spherical shell, which in turn may be surrounded by a coating that improves the abrasion resistance and storage stability of the pellets.

The preparation of pellet formulations of this kind that are preferably used according to the invention is characterised by a series of partial steps. First, the core 1 is prepared from pharmaceutically acceptable organic acid. Within the scope of the present invention tartaric acid is used to prepare the core 1. The core material 1 thus obtained is then converted into so-called isolated tartaric acid cores 3 by spraying on an isolating suspension 2. A dabigatran suspension 4 prepared subsequently is sprayed onto these coated cores 3 by means of a coating process in one or more process steps. The active substance pellets 5 thus obtained are then packed into suitable capsules.

The experimental section that follows summarises the preparation of the medicament formulations that are particularly preferably used according to the invention.

### Example 1

### Preparation of the Starter Pellets

480 kg water are heated to 50° C. and 120 kg of acacia (gum arabic) are added with stirring in a conventional mixing container having a dished end and stirrer. Stirring is continued at constant temperature until a clear solution is obtained. Once there is a clear solution (usually after 1 to 2 hours) 600 kg tartaric acid are added with stirring. The tartaric acid is added at constant temperature and while stirring is continued. After the addition has ended the mixture is stirred for about another 5 to 6 hours.

1000 kg tartaric acid are added to a slowly rotating (3 revolutions per minute) unperforated horizontal pan with a spraying and powder applying unit (e.g. Driamat 2000/2.5). Before spraying starts, a sample of the acid is taken for screening analysis. The acid in question is tartaric acid particles with a particle size in the range from 0.4-0.6 mm.

The acid rubber solution obtained by the above method is sprayed onto the tartaric acid particles thus provided. During the spraying, the quantity of air supplied is adjusted to 1000 m³/h and 35°-75° C. The differential pressure is 2 mbar and the speed of rotation of the pan is 9 revolutions per minute. The nozzles should be arranged at a distance of 350-450 mm from the filling.

The acid rubber solution is sprayed on by alternating with the following steps. After about 4.8 kg of the acid rubber solution has been sprayed onto the tartaric acid particles of particle size 0.4-0.6 mm and the solution has been distributed, about 3.2 kg tartaric acid powder are sprinkled onto the damp tartaric acid particles. The tartaric acid powder in question consists of fine tartaric acid particles with a particle size of <50 microns. In all, 800 kg tartaric acid powder are required. After the said tartaric acid powder has been sprinkled on and distributed the spray material is dried until a product temperature of about 40° C. is reached. This is in turn followed by the spraying on of the acid rubber solution.

These cycles are repeated until the acid rubber solution is used up. Once the process has ended the acid pellets are dried in the pan at 3 rpm for 240 minutes. To prevent caking after the drying has finished, an intermittent program is run at 3 rpm for 3 minutes every hour. In the present instance this means that the pan is rotated at 3 rpm for 3 minutes at intervals of one hour and then left to stand. The acid pellets are then transferred into a drying apparatus. They are then dried at 60° C. over a period of 48 hours. Finally, the particle size distribution is determined by screen analysis. The particle size with a diameter of 0.6-0.8 mm corresponds to the product. This fraction should make up >85%.

### Example 2

### Isolation of the Starter Pellets

To prepare the isolating suspension, 666.1 (347.5) kg of ethanol are placed in the mixing container and the hydroxypropylmethylcellulose (33.1 (17.3) kg) is added with stirring at approx. 600 rpm and dissolved. Then under the same conditions 0.6 (0.3) kg dimeticone are added. Shortly before use, talc (33.1 (17.3) kg) is added, again with stirring, and suspended.

The acid pellets 1200 (600) kg are poured into the coating apparatus (e.g. GS-Coater Mod. 600/Mod. 1200) and sprayed therein in the rotating pan with the isolating suspension described above in a continuous spraying process lasting several hours at a spraying rate of 32 kg/h for the 1200 kg mixture or 21 kg/h for the 600 kg mixture. The pellets are also dried continuously with an air supply at up to 70° C.

After the GS Coater has been emptied, the isolated starter pellets are fractionated by screening. The product fraction with a diameter ≦1.0 mm is stored and used further.

### Example 3

### Preparation of the Dabigatran Etexilate Suspension

26.5 kg hydroxypropylcellulose are added to 720 kg isopropanol in a 1200 litre mixing container fitted with a propeller stirrer and the mixture is stirred until fully dissolved (about 12-60 hours; roughly 500 rpm). Once the solution is clear, 132.3 kg of dabigatran etexilate methanesulphonate (polymorph I) are added with stirring (400 rpm) and the mixture is stirred for about another 20-30 minutes. Then 21.15 kg of talc is added at a constant stirring rate and stirring is continued at the same speed for about another 10-15 minutes. The steps described above are preferably carried out under a nitrogen atmosphere.

Any clumps formed are broken up by homogenising using an UltraTurrax stirrer (about 60-200 minutes). The suspension temperature should not exceed 30° C. throughout the entire manufacturing process.

The suspension is stirred until ready for further processing to ensure that no sedimentation occurs (at roughly 400 rpm).

If the suspension is stored at below 30° C., it should be further processed within at most 48 h. If for example the suspension is manufactured and stored at 22° C., it should be further processed within 60 hours.

### Example 4

### Preparation of the Dabigatran Etexilate Active Substance Pellets

A horizontal pan with an unperforated container is used (GS Coater Mod. 600). In contrast to the fluidised bed method, the suspension is sprayed onto the fluidised bed of pellets in the rotating pan by the “top spray” method. It is sprayed on through nozzles 1.4 mm in diameter. The dry air is passed into the bed of pellets through so-called immersion blades and transported away through an opening in the back wall of the coater.

The horizontal pan is charged with 320 kg of the tartaric acid pellets obtained according to Example 2 and the bed of pellets is heated up. Once a product temperature of 43° C. has been reached, spraying begins. 900 kg of the suspension prepared previously according to Example 3 are sprayed on, first of all for 2 h at a spraying rate of 20 kg/h, then 24 kg/h. The suspension is stirred constantly. The temperature of the air supplied is at most 75° C. The amount of air supplied is about 1900 m³/h.

Then the pellets are dried in the horizontal pan (5 revolutions per minute) at an air inflow temperature of at least 30° C., at most 50° C. and an air inflow amount of 500 m³/h over a period of about 1-2 hours.

325 kg of the pellets thus obtained are then loaded once more into a horizontal pan and heated to 43° C. 900 kg of the suspension prepared previously according to Example 3 are sprayed on, first of all for 2 h at a spraying rate of 20 kg/h, then 24 kg/h. The suspension is stirred constantly. The temperature of the air supplied is at most 75° C. The amount of air supplied is about 1900 m³/h.

Then the pellets are dried in the horizontal pan (5 revolutions per minute) at an air inflow temperature of at least 30° C., at most 50° C. and an air inflow amount of 500 m³/h over a period of about 1-2 hours.

The dried pellets are then passed through a vibrating screen with a mesh size of 1.6 mm and stored in containers with desiccants until needed for further processing.

### Example 5

### Examples of Formulations

The following examples of formulations are then obtained from the active substance pellets obtained according to Example 4 by packing into hydroxypropylmethylcellulose capsules:

| | amount [mg] | amount [mg] |
|---|---|---|
| Ingredient | per capsule | per capsule |
| active substance I | 86.48⁽¹⁾ | 126.83⁽²⁾ |
| Acacia (gum arabic) | 4.43 | 6.50 |
| tartaric acid | 88.56 | 129.9 |
| hydroxymethyl- | 2.23 | 3.27 |
| propylcellulose 2910 | | |
| dimethylpolysiloxane 350 | 0.04 | 0.06 |
| talc | 17.16 | 25.16 |
| hydroxypropylcellulose | 17.30 | 25.37 |
| HPMC capsule | 60⁽³⁾ | 70⁽⁴⁾ |
| Total | 276.2 | 387.1 |

⁽¹⁾corresponds to 75 mg of free active substance base
⁽²⁾corresponds to 110 mg of free active substance base
⁽³⁾weight of capsule size is about 60 mg
⁽⁴⁾weight of capsule size is about 70 mg

In another aspect the present invention relates to one of the above-mentioned medicament formulations as a secondary medication in percutaneous interventional cardiac catheterisation.

In another aspect the present invention relates to a medicament formulation which contains 60-90 mg, preferably 70-80 mg, particularly preferably about 75 mg dabigatran etexilate of formula I, as a secondary medication for percutaneous interventional cardiac catheterisation. In another aspect the present invention relates to a medicament formulation which contains 90-130 mg, preferably 100-120 mg, preferably 105-115 mg, particularly preferably about 110 mg of dabigatran etexilate of formula I as a secondary medication for percutaneous interventional cardiac catheterisation.

In another aspect the present invention relates to a medicament formulation which contains 60-90 mg, preferably 70-80 mg, particularly preferably about 75 mg dabigatran etexilate of formula I in the form of the polymorph I of its methanesulphonate as a secondary medication for percutaneous interventional cardiac catheterisation. In another aspect the present invention relates to a medicament formulation which contains 90-130 mg, preferably 100-120 mg, preferably 105-115 mg, particularly preferably about 110 mg dabigatran etexilate of formula I in the form of the polymorph I of its methanesulphonate as a secondary medication for percutaneous interventional cardiac catheterisation.

In another aspect the present invention relates to a medicament formulation which also contains hydroxymethylpropylcellulose in addition to dabigatran etexilate of formula I in the form of polymorph I of its methanesulphonate as a secondary medication for percutaneous interventional cardiac catheterisation.

In another aspect the present invention relates to a medicament formulation which also contains dimethylpolysiloxane in addition to the dabigatran etexilate of formula I in the form of polymorph I of its methanesulphonate as a secondary medication for percutaneous interventional cardiac catheterisation.

In another aspect the present invention relates to a medicament formulation which also contains the ingredients gum arabic, tartaric acid, hydroxymethylpropylcellulose, dimethylpolysiloxane, talc and hydropropylcellulose in addition to the dabigatran etexilate of formula I in the form of polymorph I of its methanesulphonate as a secondary medication for percutaneous interventional cardiac catheterisation.

In another aspect the present invention relates to a medicament formulation which exclusively contains the ingredients gum arabic, tartaric acid, hydroxymethylpropylcellulose, dimethylpolysiloxan, talc and hydropropylcellulose in addition to dabigatran etexilate of formula I in the form of polymorph I of its methanesulphonate as a secondary medication for percutaneous interventional cardiac catheterisation.

In another aspect the present invention relates to a method of carrying out percutaneous interventional cardiac catheterisation, characterised in that dabigatran etexilate of formula I is used, optionally in the form of the tautomers, pharmaceutically acceptable salts, polymorphs, solvates or hydrates thereof.

In another aspect the present invention relates to a method of carrying out percutaneous interventional cardiac catheterisation, characterised in that dabigatran etexilate of formula I is used in the form of one of the above-mentioned medicament formulations.

1. A method to prevent coagulation in a patient undergoing percutaneous, interventional cardiac catheterization comprising the step of administering to the patient a compound of formula II 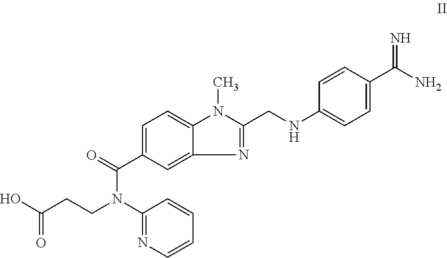
optionally in the form of the tautomers, the pharmaceutically acceptable salts or prodrugs thereof prior to or during said catheterization.

2. The method according to claim 1, wherein the prodrug of the compound of formula II is the compound of formula I 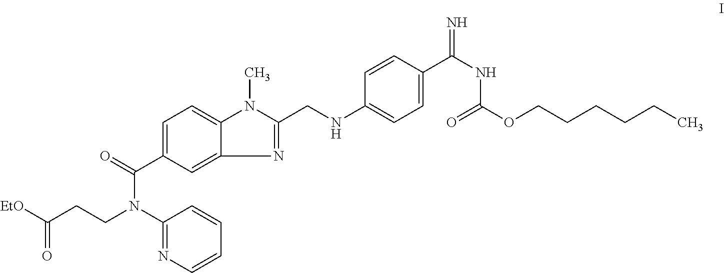
optionally in the form of the tautomers and the pharmaceutically acceptable salts thereof.

3. The method according to claim 2, wherein between 50 and 400 mg of the compound of formula I is administered per day.

4. The method according to one of claims 2-3, wherein the pharmaceutically acceptable salts include acid addition salts which are selected from among the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydrolactate, hydroxalate, hydrosuccinate, hydrobenzoate and hydro-p-toluenesulphonate.

5. The method according to claim 4, wherein the pharmaceutically acceptable salts include is selected from hydrochloride, hydrobromide, hydrosulphate, hydrophosphate, hydromaleate, hydrofumarate and hydromethanesulphonate.

6-8. (canceled)

9. A method to prevent coagulation in a patient undergoing percutaneous, interventional cardiac catheterization comprising the step of administering to the patient a pharmaceutical composition comprising a compound of formula I 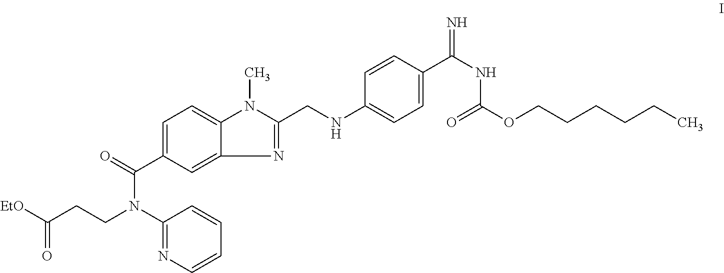
optionally in the form of the tautomers and the pharmaceutically acceptable salts thereof, prior to or during said catheterization.

10. The method according to claim 3, wherein between 75 to 350 mg of the compound of formula I is administered per day.

11. The method according to one of claims 2-3, wherein the pharmaceutically acceptable salt is hydrochloric acid, methanesulphonic acid, maleic acid, benzoic acid or acetic acid.

12. The method according to one of claims 2-3, wherein the pharmaceutically acceptable salt is methanesulphonic acid or mesylate.
